(19) United States
(12) Patent Application Publication
(10) Pub. No.: US 2022/0240993 A1
(43) Pub. Date: Aug. 4, 2022

(54) Backout Resistant Screw

(71) Applicant: CTL Medical Corporation, Addison, TX (US)

(72) Inventors: Rob TREZISE, Coatesville, PA (US); Sean SUH, Milltown, NJ (US); Jon SUH, Ambler, PA (US)

(21) Appl. No.: 17/665,077

(22) Filed: Feb. 4, 2022

Related U.S. Application Data
(60) Provisional application No. 63/145,914, filed on Feb. 4, 2021.

(51) Int. Cl. A61B 17/86

(52) U.S. Cl. CPC A61B 17/863; A61B 17/8615

(57) ABSTRACT

Disclosed are devices, systems and/or methods for use in the surgical treatment of vertebrae and/or other bones, particularly bone screws having features and/or attributes that allow secure fixation of the device to the bone and prevention and/or inhibition of undesirable loosening and/or “back-out” of the screw body from a targeted surgical site.

### CROSS-REFERENCE TO RELATED APPLICATIONS

This application claims priority to and benefit of U.S. Provisional Patent Application No. 63/145,914 entitled “Anti-Backout Screw” filed Feb. 4, 2021, the disclosure of which is incorporated by reference herein in its entirety.

### FIELD OF THE INVENTION

The present invention relates generally to the field of medical devices. In particular, the present subject matter relates to fasteners such as bone screws having features and/or attributes that allow secure fixation of the device to the bone and prevents and/or inhibits undesirable loosening and/or “back-out” of the screw body from the targeted surgical site.

### BACKGROUND OF THE INVENTION

The spinal column of vertebrates provides support to bear weight and protection to the delicate spinal cord and spinal nerves. The spinal column includes a series of vertebrae stacked on top of each other. There are typically seven cervical (neck), twelve thoracic (chest), and five lumbar (low back) segments. Each vertebra has a cylindrical shaped vertebral body in the anterior portion of the spine with an arch of bone to the posterior, which covers the neural structures. Between each vertebral body is an intervertebral disk, a cartilaginous cushion to help absorb impact and dampen compressive forces on the spine. To the posterior, the laminar arch covers the neural structures of the spinal cord and nerves for protection. At the junction of the arch and anterior vertebral body are articulations to allow movement of the spine.

Various types of problems can affect the structure and function of the spinal column. These can be based on degenerative conditions of the intervertebral disk or the articulating joints, traumatic disruption of the disk, bone or ligaments supporting the spine, tumor or infection. In addition, congenital or acquired deformities can cause abnormal angulation or slippage of the spine. Anterior slippage (spondylolisthesis) of one vertebral body on another can cause compression of the spinal cord or nerves. Patients who suffer from one of more of these conditions often experience extreme and debilitating pain and can sustain permanent neurological damage if the conditions are not treated appropriately.

Various physical conditions can manifest themselves in the form of damage or degeneration of an intervertebral disc, the result of which is mild to severe chronic back pain. Intervertebral discs serve as “shock” absorbers for the spinal column, absorbing pressure delivered to the spinal column. Additionally, they maintain the proper anatomical separation between two adjacent vertebrae. This separation is necessary for allowing both the afferent and efferent nerves to exit and enter, respectively, the spinal column. Alternatively, or in addition, there are several types of spinal curvature disorders. Examples of such spinal curvature disorders include, but need not be limited to, lordosis, kyphosis and scoliosis.

Various techniques for treating spinal disorders or other bone structures can include the use of anchoring and/or connecting devices such as screws or other fasteners, which are typically secured into and/or through one or more bone structures for a variety of reasons. While rotation of a screw desirably advances and secures the screw threads within the bone structure, the is a significant potential post-surgery for the screw to rotate in an opposing direction, thereby “loosening,” withdrawing and/or otherwise causing the screw to exit the bone structure—which can be highly undesirable in many situations, especially long after completion of a surgical procedure. Accordingly, there is need for further improvement in surgical implants, and the present subject matter is such improvement.

### BRIEF SUMMARY OF THE INVENTION

The following presents a simplified summary of the subject matter in order to provide a basic understanding of some aspects of the subject matter. This summary is not an extensive overview of the subject matter. It is intended to neither identify key or critical elements of the subject matter nor delineate the scope of the subject matter. Its sole purpose is to present some concepts of the subject matter in a simplified form as a prelude to the more detailed description that is presented later.

In accordance with various aspects of the present subject matter, the present invention is directed, in a particular aspect, to a bone screw having a head and a shaft portion, wherein some portion of the screw includes one or more rotation inhibiting portions which desirably allow the screw to be advanced to an intended position, location and/or orientation within a bony structure, but which subsequently “locks” or otherwise inhibits subsequent rotation of the screw in an undesired fashion, including screw rotation and/or micromotion which tends to loosen or remove the screw from the targeted bone location.

In at least one exemplary embodiment, a bone screw can include a shaft having a proximal neck section and a distal section, the proximal neck section having a first thread form disposed thereon and the distal section having a second thread form or similar structure disposed thereon. The thread profile of the first and second thread forms is desirably different in some manner, and in various embodiments the first thread form can comprise a left-handed thread, and the second thread form can comprise a right-handed thread. In some embodiments, the first thread form can be embedded into the shaft portion, with the second thread form extending outward from the shaft portion.

In some embodiments, one or more surfaces of the screw, optionally including the one or more rotation inhibiting portions, can include a bone growth promoting structure or coating, such as a surface which incorporates silicon nitride (i.e., Si₃N₄ and/or chemical analogues thereof) in its construction, either in the entirety of the structure as well as components, portions, layers and/or surface coatings thereof. In various embodiments, such silicon nitride material(s) can be highly osteo-inductive and/or osteoconductive and will desirably facilitate and/or promote implant fixation to adjacent living bone surfaces, while concurrently reducing and/or inhibiting periprosthetic infection and/or bacterial adhesion to the surfaces and/or interior portions of the implant.

In various applications, the utility of silicon nitride as an implant material can be enhanced by the addition of various other medical materials, including the use of one or various combinations of titanium, chrome cobalt, stainless steel, silicone, poly (ether ether ketone) (PEEK), ultra-high molecular-weight polyethylene (UHMWPE), polyurethane foams, polylactic acid, apatites and/or various 3D printed materials. In such cases, the employment of such material mixtures in implant construction may enhance the strength and/or durability of a desired implant design, as well as allow for improved surgical outcomes and/or greatly reduced complication rates.

In accordance with another aspect of the present subject matter, various methods for manufacturing devices and/or components thereof, as set for within any of the details described with the present application, are provided.

While embodiments and applications of the present subject matter have been shown and described, it would be apparent that other embodiments, applications and aspects are possible and are thus contemplated and are within the scope of this application.

The following description and the annexed drawings set forth in detail certain illustrative aspects of the subject matter. These aspects are indicative, however, of but a few of the various ways in which the principles of the subject matter may be employed and the present subject matter is intended to include all such aspects and their equivalents. Other objects, advantages and novel features of the subject matter will become apparent from the following detailed description of the subject matter when considered in conjunction with the drawings.

### BRIEF DESCRIPTION OF THE SEVERAL VIEWS OF THE DRAWINGS

The foregoing and other features and advantages of the present subject matter will become apparent to those skilled in the art to which the present subject matter relates upon reading the following description with reference to the accompanying drawings.

FIG. 1 depicts a perspective view of one exemplary embodiment of a fastener constructed in accordance with various teachings herein;

FIG. 2 illustrates an alternative perspective view of the fastener of FIG. 1;

FIG. 3 depicts a top plan view of the fastener of FIG. 1;

FIGS. 4 and 5 depict left side and right side views, respectively, of the fastener of FIG. 1;

FIG. 6 depicts a perspective view of a backout resistant fastener;

FIG. 7 depicts a perspective view of a backout resistant fastener incorporating various anti-rotation components;

FIG. 8 depicts one alternative embodiment of a fixation screw which incorporates a plurality of fenestrations or openings in a side wall of the shaft;

FIG. 9 depicts rotation of the screw of FIG. 8 to draw the screw shaft into the bony structure or substrate;

FIG. 10 depicts the screw of FIGS. 8 and 9 in a final desired position with the anti-backout structures press-fit into the bony structure or substrate; and

FIG. 11 depicts an exemplary sacral fixation using a fixation screw with anti-backout features described herein.

### DETAILED DESCRIPTION OF THE INVENTION

The disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure. Moreover, it is noted that like reference numerals represent similar parts throughout the several views of the drawings.

The terms “including,” “comprising” and variations thereof, as used in this disclosure, mean “including, but not limited to,” unless expressly specified otherwise. The terms “a,” “an,” and “the,” as used in this disclosure, mean “one or more,” unless expressly specified otherwise.

Devices and/or device components that are disclosed in communication with each other need not necessarily be in continuous communication with each other, unless expressly specified otherwise. In addition, devices that are in direct contact with each other may contact each other directly or indirectly through one or more intermediary articles or devices.

The disclosed screws may be constructed of medical grade titanium, which term is meant to encompass both titanium and titanium alloys, and in various embodiments may be grit blasted to smooth the comers and edges. In other embodiments, the devices disclosed herein may comprise different materials and/or combinations of materials. The materials may comprise metals, polymers, and/or ceramics. The metals may comprise of titanium, steel, tantalum, cobalt-chrome, cobalt-chrome alloys, titanium alloy, nitinol and/or any combination thereof. Polymeric materials may comprise PEEK. Ceramic materials may comprise alumina, zirconia, silicon nitride, vitoss bone graft substitute, vitrium, and/or any combination thereof. Alternatively, the device(s) disclosed herein may be made of a material such as a polymer, a metal, an alloy, or the like, including titanium, a titanium alloy, or the like, or various combinations of the foregoing. In various optional embodiments, the disclosed devices may include other materials such as, for example, silicon nitride, which may be optionally in combination with a Polyether Ether Ketone (PEEK), titanium, a titanium alloy, or the like, or various combinations of the foregoing.

In various embodiments, the device may include one or more surface features or other components formed by a process such as, for example, an active reductive process of a metal (e.g., titanium or titanium alloy) to increase an amount of a nanoscaled texture to device surface(s), so as to increase promotion of bone growth and fusion.

Although process steps, method steps, or the like, may be described in a sequential order, such processes and methods may be configured in alternate orders. In other words, any sequence or order of steps that may be described does not necessarily indicate a requirement that the steps be performed in that order. The steps of the processes or methods described herein may be performed in any order practical. Further, some steps may be performed simultaneously.

When a single device or article is described herein, it will be readily apparent that more than one device or article may be used in place of a single device or article. Similarly, where more than one device or article is described herein, it will be readily apparent that a single device or article may be used in place of the more than one device or article. The functionality or the features of a device or article may be alternatively embodied by one or more other devices or articles which are not explicitly described as having such functionality or features.

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention provides various devices, systems and methods for treating various anatomical structures of the spine and/or other areas of human and/or animal bodies. While the disclosed embodiments may be particularly well suited for use during surgical procedures for the repair, fixation and/or support of vertebrae, it should be understood that various other anatomical locations of the body may benefit from various features of the present invention.

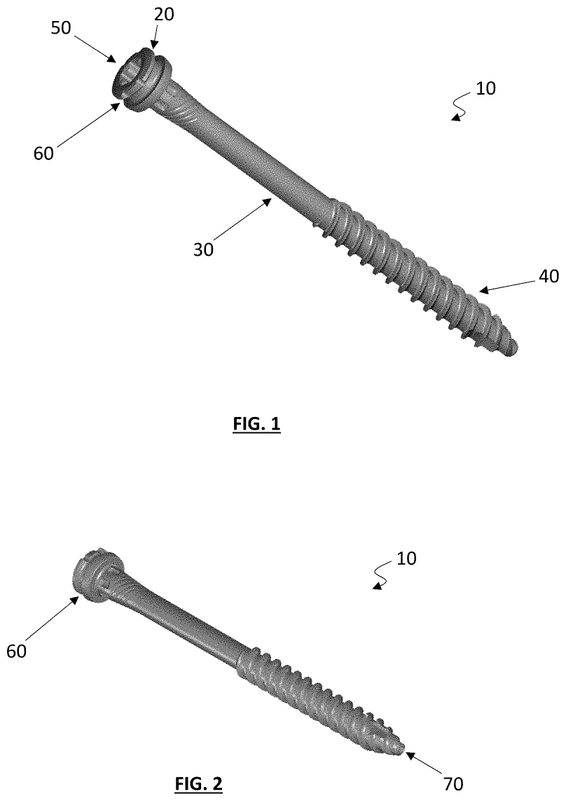

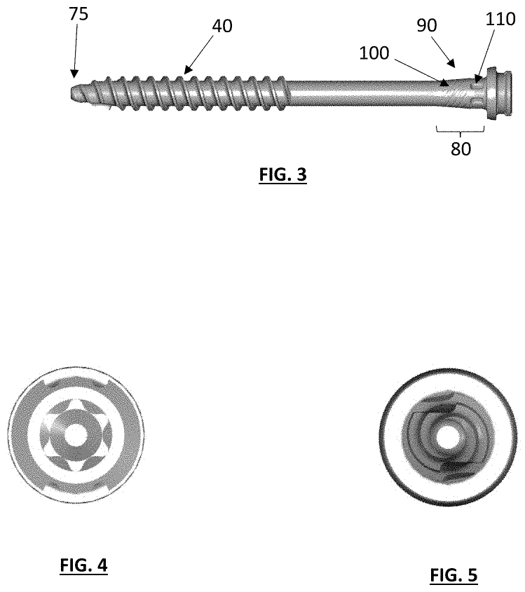

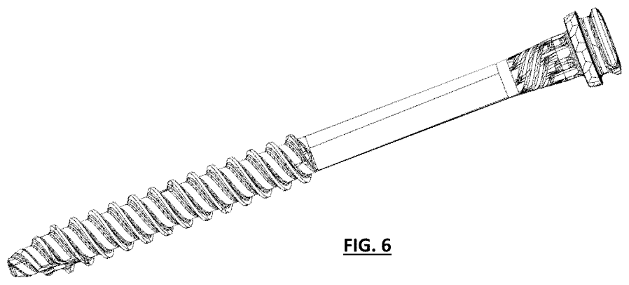

FIG. 1 depicts a fixation screw 10 comprising a head 20, a shaft 30 and a thread 40. If desired, the fixation screw may further include a proximal collar (not shown) near the head. In some embodiments, the fixation screw may include self-tapping features such as at least a portion of the screw length having a fine or coarse self-tapping thread. The length of the threaded portion of the screw is suitably from 10 to 50 mm, although other lengths may be suitable for various targeted anatomical regions and/or surgical procedures. It is generally suitable to manufacture screws in various sizes such that the length of the said threaded portion varies so different sized screws can be chosen for any particular application, with a kit of multiple screws of varying shapes and/or sizes being provided for a surgeon's use.

If desired, the head may include one or more driving features such as a driving tool recess 50, wherein the driving tool recess 50 may comprise a shape that is sized and configured to receive a slotted driver, a cruciform driver, external polygon driver, internal polygon driver, hexalobular driver, three-pointed driver, special driver and/or any combination thereof. In the current embodiment, a hexagonal wrenching counterbore is formed into a slightly enlarged rear end for driving the screw.

Optionally, a channel 60 may be disposed on the circumference of the head of the screw. The channel may be sized and configured to receive a portion of a collar or similar component, or the channel may be configured for attachment as a subcomponent of a larger surgical construct.

The screw shaft and thread may have a diameter. The shaft diameter may be smaller and/or greater than the thread.

In various embodiments, the screw 10 may include a cannulation 70 or fenestration through an entirety or some portion of the shaft.

As best seen in FIG. 23, the thread 40 which desirably secures the screw to a targeted anatomical region can be positioned near a distal end 75 of the shaft 30 (in a known manner). A proximal end 80 of the shaft 30, however, can desirably include one or more structural features which desirably inhibit rotation and/or loosening of the screw from the targeted anatomical region after completion of the surgical procedure. For example, a distal or lead end 80 of the shaft may be of a slightly smaller diameter, i.e., 0.05 inches smaller than the proximal shaft end, thereby allowing the implant to be fully seated with the anti-rotation features engaging the surrounding bone tissues. The proximal end 80 of the shaft can desirably incorporate an increased diameter section 90 (which in this embodiment includes a tapered section gently increasing in diameter), with some or all of this section 90 incorporating one or more cups or depressions 110 and/or one or more counter-helical grooves 100 and/or similar reverse threaded features.

In various embodiments, the grooves may be relatively wide (i.e., up to and/or exceeding 0.029 inches) and may also be quite shallow (i.e., approximately 0.013 inches), with the dimensions of the grooves possibly varying slightly relative to the diameter of the implant and/or localized thickness of the increased diameter and/or tapered regions. If desired, the helical groove(s) and/or depressions/cups may be machined into a cylindrical surface portion of the screw, desirably leaving much of the remaining cylindrical surface mostly uninterrupted to predominate the total exterior area of the shaft.

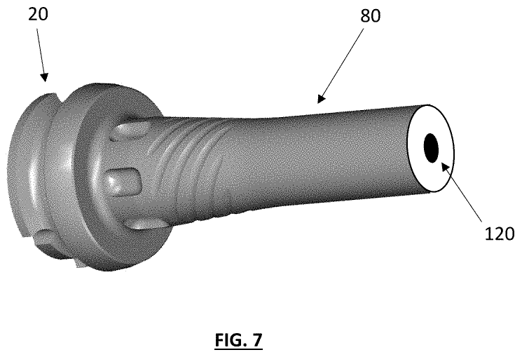

FIG. 7 depicts a partial enlarged view of the head 20 and proximal end 80 of the shaft 30 of FIG. 3, with the cutaway shaft 30 showing a centrally positioned cannulated opening 120. A series of cups or depressions 110 are positioned on an outer periphery of the increased diameter shaft section 90. In addition, a reverse thread or counter helical grooved portion 100 is formed into the increased diameter shaft section 90. During placement of the screw, rotation of the screw in a first direction (i.e., clockwise) desirably rotates the shaft causing the threads to advance into the targeted anatomy and draws the screw into the bone. Continued rotation of the screw will eventually draw the proximal end 80 of the screw into the bone, with the increased diameter shaft section 90 desirably being pulled and/or press-fit into the opening and/or causing bone structures to wedge into the outer surfaces of the cutaway bone passage (not shown). Desirably, this will cause bone fragments and/or bone structures to compact and/or press into the depressions 110 and/or grooves of the reverse threaded feature. Desirably, these structures will inhibit counterrotation of the screw in the opposing direction (i.e., counterclockwise), thereby inhibiting and/or preventing loosening of the screw in an undesired manner. In addition to providing immediate resistance to counterrotation, one or more of these structures will desirably provide increased resistance to screw loosen as bony structures adhere and/or grow into these features—desirably preventing loosening of the screw over time from micromotion and/or subsidence.

The presence of the grooves and/or depressions/cups creates a slight threaded engagement when the body 12 is press fit for improved retention of the implant, since the bone tissue will protrude slightly into the groove 14. The groove 14, being a curved rounded shape and arcuate in section, promotes the growth of bone tissue into the groove 14 for permanent, secure implant retention.

In at least one exemplary embodiment, a series of pitched helical grooves (or series of groove portions) can be provided that are partially and/or fully recessed into the smooth cylindrical surface of the implant body, including being recessed into an enlarged or tapered portion of a screw shaft. In various embodiments, the groove location and/or shape can promote rapid bone growth into the groove and the helical shape desirably creates a slight threaded retention as the adjacent bone tissue will desirably protrude slightly into the groove under the pressure of the implant. Desirably, the shallow groove/depression depth can easily accept surrounding bone tissue as the screw is advanced into the bone, with displaced bone fragments desirably guided into the grooves and/or depressions. The shallowness of the grooves and/or depressions desirably obviates the need for any special hole preparation and avoids imposition of significant stress on the bone tissue to insure long term secure retention of the screw. If desired, one or more long pitched helical grooves may be machined into a tapered and/or enlarged portion of the shaft, wherein the turns may be spaced so that much of the length of the shaft is constituted by a cylindrical surface. The grooves may be arcuate in section and gently curved due to the long pitch.

In various embodiments, one or more shallow pitch threads may alternatively be added to central and/or distal structures of the implant, including areas possibly having threads projecting from the cylindrical surface of the body of the implant, which may have an opposite helix angle from the helical groove turns but of approximately the same long pitch. In an optional arrangement the distal thread turns may be interposed between one or more distal groove turns so as to cross the groove turns at diametrically opposite points on the implant body. Segments of the thread can be removed in the areas where the thread turns cross the grooves to create a more flattened and/or recessed shape.

It should be understood that, in alternative embodiments, the grooves and/or depressions may be positioned distal from, adjacent to and/or overlapping some or all of the screw threads. For example, the turns of the screw thread may be disposed intermediate the turns of the groove(s). In such a case, when the groove is machined, the screw threads may be eliminated in the crossing areas, if desired.

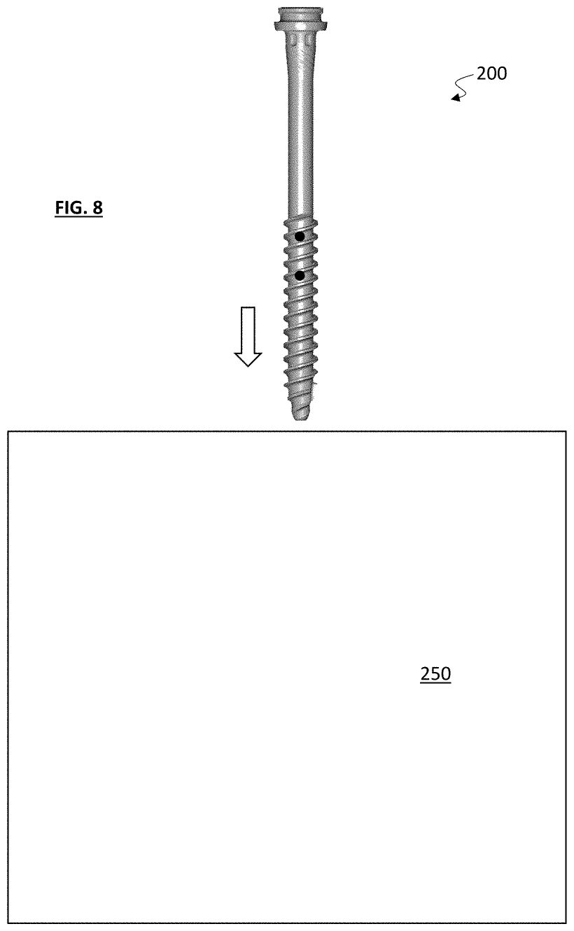

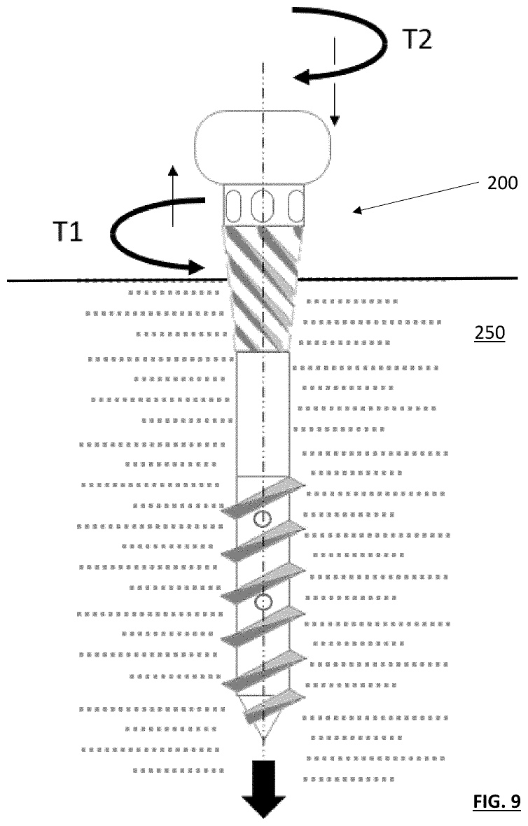

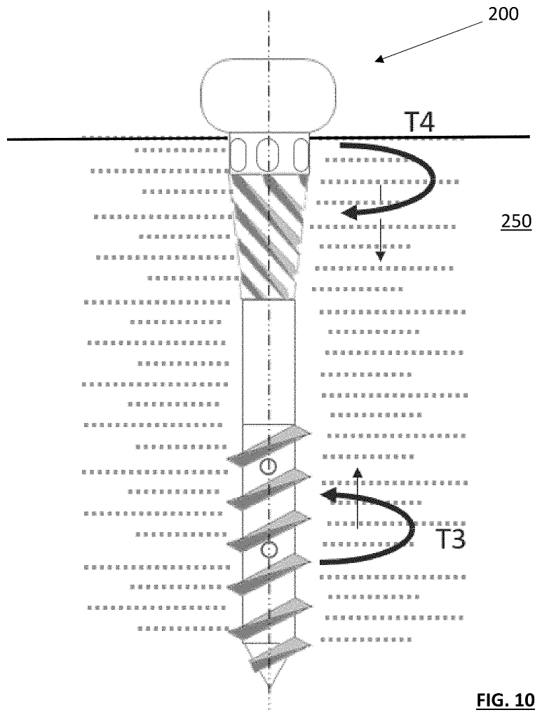

FIG. 8 depicts one alternative embodiment of a fixation screw 200 which incorporates a plurality of fenestrations or openings 210 positioned between flutes of a screw thread 220. In this embodiment, rotation of the fixation screw 200 desirably causes the screw threads 220 to engage with a bony structure 250, causing the screw to advance into the bony structure 250 in a known manner. As best seen in FIG. 9, continued rotation of the screw 200 draws the screw shaft into the bony structure 250, with the clockwise rotation force T2 causing insertion of the screw 200 is much greater than the counterclockwise resistance force T1 from the anti-backout structures.

FIG. 10 depicts the screw 200 and bony structure 250 of FIG. 9, but with the screw 200 in a final desired position where some and/or all of the anti-backout structures are in contact with and/or are fully seated into the bony structure. In this embodiment, the torsional resistance force T4 (caused by the anti-backout structures) is desirably significantly greater than the backing-out force T3 (which is caused by movement and micromotion between of the surrounding anatomy and the screw). Because T4 is equal to or greater than T3, the screw desirably remains within a desired position with the bone and does not loosen.

The counter-wound relationship of the groove and screw thread desirably causes bone fragments displaced by the thread to be pushed into the grooves/depressions when the screw is advanced sufficiently into the bone. The screw threads desirably provide an immediate mechanical holding force upon advancing the screw into position. In various embodiments, the screw may be a self-tapping type of screw, or a passage may be formed in the bony tissue prior to placement and/or advancement of the screw therein.

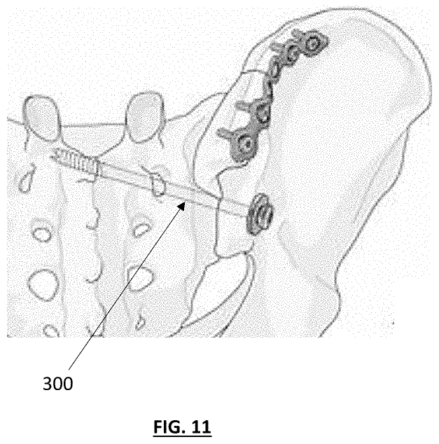

In various embodiments the disclosed fixation devices may be particularly useful to prevent loosening and/or back-out of fixation screws which experience prolonged exposure to micro-motion that may be present in a joint or pseudo joint structure, such as where the fixation device comprises a lag-type screw 300 utilized in fixation of a sacral anatomy (see FIG. 11). Desirably, once the fixation device is properly positioned in a desired manner, anatomical forces will then be unlikely and/or unable to introduce micromotion and facilitate the backing out of the fixation screw assembly.

In various embodiments, a substrate into which the screw may be advanced may comprise a bone, an implant, and/or a tissue. Suitable implants structures that may be utilized with the disclosed fastening devices may include a plate, a disc, a cage, a fusion rod construct and/or any combinations thereof.

In various embodiments, a fixation screw assembly may further comprise a bone growth material and/or member, such as an attachable modular component comprising a semi-cylinder or half-cylinder which can be placed onto and/or around a screw shaft. An inner diameter of the bone growth member may match or substantially match an outer diameter of the screw shaft, with the bone growth member optionally disposed onto the shaft. The length of the bone growth member may match or substantially match the length of the shaft. The length of the bone growth member may be smaller the length of the shaft. The outer diameter of the bone growth member may comprise a texture and/or features. The texture and/or features may comprise macro-, micro-, and nanometer sized textures and/or features. The textures and/or features may further include ribs, grooves, protrusions, threads or threading, sandblasting, laser etching, acid etching, anodization, and/or any combination thereof. The outer diameter further may comprise a coating, the coating may comprise plasma coating, bioactive coatings, antibiotic coatings, growth factor coatings, anticoagulation coating, bone remodeling agents, bacteriostatic coating (e.g., silicon nitride or Si3N4) and/or any combination thereof.

In various embodiments, a surface, a depression, a groove and/or a modular component of a fixation screw may comprise silicon nitride (Si3N4) and its various analogs, which can impart both antibacterial and osteogenic properties to an implant, including to bulk Si3N4 as well as to implants coated with layers of Si3N4 of varying thicknesses. In bone replacement as well as prosthetic joint fusion and/or replacement, osseous fixation of implants through direct bone ingrowth (i.e., cementless fixation) is often preferred, and such is often attempted using various surface treatments and/or the incorporation of porous surface layers (i.e., porous Ti6Al4V alloy) on one or more bone-facing surfaces of an implant. Silicon nitride surfaces express reactive nitrogen species (RNS) that promote cell differentiation and osteogenesis, while resisting both gram-positive and gram-negative bacteria. This dual advantage of RNS in terms of promoting osteogenesis, while discouraging bacterial proliferation, can be of significant utility in a variety of implant designs.

Desirably, the inclusion of silicon nitride components into a given screw or implant design could encompass the use of bulk silicon nitride implants, as well as implants incorporating other materials that may also include silicon nitride components and/or layers therein, with the silicon nitride becoming an active agent of bone fusion. RNS such as N2O, NO, and —OONO are highly effective biocidal agents, and the unique surface chemistries of Si3N4 facilitate its activity as an exogenous NO donor. Spontaneous RNS elution from Si3N4 discourages surface bacterial adhesion and activity, and unlike other direct eluting sources of exogenous NO, Si3N4 elutes mainly NH4+ and a small fraction of NH3 ions at physiological pH, because of surface hydrolysis and homolytic cleavage of the Si—N covalent bond. Ammonium NH4+ can enter the cytoplasmic space of cells in controlled concentrations and through specific transporters, and is a nutrient used by cells to synthesize building-block proteins for enzymes and genetic compounds, thus sustaining cell differentiation and proliferation. Together with the leaching of orthosilicic acid and related compounds, NH4+ promotes osteoblast synthesis of bone tissue and stimulates collagen type 1 synthesis in human osteoblasts. Conversely, highly volatile ammonia NH3 can freely penetrate the external membrane and directly target the stability of DNA/RNA structures in bacterial cells. However, the release of unpaired electrons from the mitochondria in eukaryotic cells activates a cascade of consecutive reactions, which starts with NH3 oxidation into hydroxylamine NH₂OH (ammonia monooxygenase) along with an additional reductant contribution leading to further oxidation into NO2- nitrite through a process of hydroxylamine oxidoreductase. This latter process involves nitric oxide NO formation. In Si3N4, the elution kinetics of such nitrogen species is slow but continuous, thus providing long-term efficacy against bacterial colonies including mutants (which, unlike eukaryotic cells, lack mitochondria). However, when slowly delivered, NO radicals have been shown to act in an efficient signaling pathway leading to enhanced differentiation and osteogenic activity of human osteoblasts. Desirably, Si3N4 materials can confer resistance against adhesion of both Gram-positive and Gram-negative bacteria, while stimulating osteoblasts to deposit more bone tissue, and of higher quality.

Where the presence of bulk silicon nitride implant materials may not be desired and/or may be impractical for some reason, it may be desirous to incorporate modules and/or layers (such as surface and/or subsurface layers and/or fillings) including silicon nitride on other materials. Silicon nitride structures and/or components can be formed using a variety of techniques, including by compressing, milling and firing silicon nitride powder, as well as by extruding silicon nitride into sheet, tube, pipe and/or thread form (which may be further processed into thread or “rope” by braiding and/or other techniques). Silicon nitride shapes may also be manufactured using subtractive manufacturing techniques (i.e., machining, milling and/or surface roughening), as well as by using additive manufacturing techniques (i.e., surface coating, brazing, welding, bonding, deposition on various material surfaces and/or even by 3D laser printing of structures). If desired, silicon nitride may even be formed using curing or other light/energy activation techniques, such as where a slurry of liquid polymer and silicon nitride particles may be UV cured to create a 3-dimensional structure and/or layer containing silicon nitride. In various embodiments, silicon nitride may be utilized in block form, in sheets, columns and bars, in cable or braided form, in mesh form, in a textured surface coating, in powder form, in granular form, in gel, in putty, in foams and/or as a surface filler and/or coating. In some cases, a surface layer of silicon nitride may be formed on an external and/or internal surface of an implant.

For example, in some embodiments it may be desirous to laser-sinter a thin layer of silicon nitride material (i.e., powder) to the surface of another material, such as PEEK or titanium. One exemplary starting micrometric powder used for laser-sintering of a Si3N4 coating in this manner could comprise a 90 wt % fraction of Si3N4 powder mixed with a 6 wt % of yttrium oxide (Y2O3) and a 4 wt % of aluminum oxide (Al2O3). If desired, a Vision LWI VERGO-Workstation equipped with a Nd:YAG laser with a wavelength of 1064 nm (max pulse energy: 70 J, peak power 17 kW, voltage range 160-500 V, pulse time 1-20 ms, spot size 250-2000 μm) can be utilized to achieve densification of successive layers of Si3N4 powder placed on a water-wet surface of a Titanium substrate in a nitrogen environment, which desirably limits Si3N4 decomposition and oxidation. In the exemplary embodiment, the Nd:YAG laser can be pulsed with a spot size of 2 mm, and driven by an applied voltage of 400 V with a pulse time of 4 ms. This operation can be repeated until a continuous thickness of 15 μm (±5 μm) is formed over an entire surface of the Titanium substrate. This process can create a wavy morphology of the ceramic/metal interface, with interlocks at the micrometer scale between metal and ceramic phases and desirably little or no diffusional transport of the Titanium element into the coating during laser sintering.

Where the presence of bulk silicon nitride implant materials may not be desired and/or may be impractical for some reason, it may be desirous to incorporate modules and/or layers including silicon nitride on other materials. Silicon nitride structures and/or components can be formed using a variety of techniques, including by compressing, milling and firing silicon nitride powder, as well as by extruding silicon nitride into sheet, tube, pipe and/or thread form (which may be further processed into thread or “rope” by braiding and/or other techniques). Silicon nitride shapes may also be manufactured using subtractive manufacturing techniques (i.e., machining, milling and/or surface roughening), as well as by using additive manufacturing techniques (i.e., surface coating, brazing, welding, bonding, deposition on various material surfaces and/or even by 3D laser printing of structures). If desired, silicon nitride may even be formed using curing or other light/energy activation techniques, such as where a slurry of liquid polymer and silicon nitride particles may be UV cured to create a 3-dimensional structure and/or layer containing silicon nitride. In various embodiments, silicone nitride may be utilized in block form, in sheets, columns and bars, in cable or braided form, in mesh form, in a textured surface coating, in powder form, in granular form, in gel, in putty, in foams and/or as a surface filler and/or coating. In some cases, a surface layer of silicon nitride may be formed on an external and/or internal surface of an implant.

For example, in some embodiments it may be desirous to laser-sinter a thin layer of silicon nitride material (i.e., powder) to the surface of another material, such as PEEK or titanium. One exemplary starting micrometric powder used for laser-sintering of a Si₃N₄ coating in this manner could comprise a 90 wt % fraction of Si₃N₄ powder mixed with a 6 wt % of yttrium oxide (Y₂O₃) and a 4 wt % of aluminum oxide (Al₂O₃). If desired, a Vision LWI VERGO-Workstation equipped with a Nd:YAG laser with a wavelength of 1064 nm (max pulse energy: 70 J, peak power 17 kW, voltage range 160-500 V, pulse time 1-20 ms, spot size 250-2000 μm) can be utilized to achieve densification of successive layers of Si₃N₄ powder placed on a water-wet surface of a Titanium substrate in a nitrogen environment, which desirably limits Si₃N₄ decomposition and oxidation. In the exemplary embodiment, the Nd:YAG laser can be pulsed with a spot size of 2 mm, and driven by an applied voltage of 400 V with a pulse time of 4 ms. This operation can be repeated until a continuous thickness of 15 μm (±5 μm) is formed over an entire surface of the Titanium substrate. This process can create a wavy morphology of the ceramic/metal interface, with interlocks at the micrometer scale between metal and ceramic phases and desirably little or no diffusional transport of the Titanium element into the coating during laser sintering.

In various embodiments, the properties of the disclosed fixation screws will desirably include improvements in one or more of the following: (1) Flexibility in manufacturing and structural diversity, (2) Strong, tough and reliable constructs, (3) Phase stable materials, (4) Favorable imaging characteristics, (5) Hydrophilic surfaces and/or structures, (6) Osteoconductive, (7) Osteoinductive, and/or (8) Anti-Bacterial characteristics.

In some surgical situations, a medical screw or other implant may often be accompanied by surgical bone defects that do not fill in with new bone over time, as well as potential infection sites proximate to the screw/implant that may be difficult or impossible to resolve (potentially necessitating implant removal in some cases). In a similar manner, bone infection sites near titanium implants can also be difficult or impossible to resolve, and may similarly necessitate implant removal. However, with a screw or implant comprising, at least in part, silicon nitride, a surface chemistry of the screw or implant may actively destroy infectious bacterial agents, and also induces new bone growth immediately upon implantation. In essence, the effect of the silicon nitride material on new bone growth acts like a magnet on ferrous materials, actively “drawing” new bone near and into the screw or implant.

Another significant advantage of using silicon nitride materials in bone implants is the anti-bacterial effects of the material on infectious agents. Upon implantation a silicon nitride surface can induce an inflammatory response action which attacks bacterial biofilms near the implant. This reaction can also induce the elevation of bacterial pods above the implant surface by fibrin cables. Eventually the bacteria in the vicinity of the silicon nitride implant surfaces will be cleared by macrophage action, along with the formation of osteoblastic-like cells. In various experiments involving comparisons between standard implants and silicon nitride implants (both bulk and silicon nitride coated implants of standard materials), cell viability data in (which were determined at exposure times of 24 and 48 hours) showed the existence of a larger population of bacteria on the standard medical materials as compared to Si₃N₄ implants (both coated and bulk). A statistically validated decreasing trend for the bacterial population with time was detected on both coated and bulk substrates, with a highest decrease rate on Si₃N₄-coated substrates. Moreover, the fraction of dead bacteria at 48 h was negligible on the standard implants, while almost the totality of bacteria underwent lysis on the Si₃N₄ substrates. In addition, optical density data provided a direct assessment of the high efficacy of the Si₃N₄ surfaces in reducing bacterial adhesion.

In various embodiments, silicon nitride materials can be incorporated into a variety of screws, implants and implant-like materials, including (1) orthopedic bone fusion implants (i.e., screws, cages, cables, rods, plugs, pins), (2) dental implants, (3) cranial/maxillofacial implants, (4) extremity implants, (5) hip and joint implants, (6) bone cements, powders, putties, gels, foams, meshes, cables, braided elements, and (7) bone anchoring elements and/or features. Where a surface coating of silicon nitride is added to an existing implant, such as to a titanium implant using a 3D-laser-sintering manufacturing process of deposition, this surface coating may comprise a dense, tenaciously adherent Si₃N₄ coating (with thickness 10-20 μm) onto the porous T-alloy surface of commercially available components, which may achieve rapid osseous fixation, while resisting bacteria. In various embodiments, the disclosed grooves and/or depressions/cups may include one or more silicon nitride surfaces and/or portions therein/thereof.

Because many forms of silicon nitride exhibit ceramic-like mechanical properties, these materials may not be well suited for use in screws that may be more than 4 mm in diameter and 15 mm in length, which can be subject to various brittleness failures when inserted into a bone. For spinal applications, where bigger diameter screws such as up to 10.5 mm in diameter and lengths up to 120 mm long may be required, more traditional implants of metal may be desirous for implantation, such as to overcome friction and hardness of human/animal bone. Thus, a typical screw consisting of a single material, screw head, threaded shaft, and tapered tip with cutting flutes may desirably be reconfigured where the threaded shaft portion or various components thereof is partially made of or incorporates a bone-growth enhancing non-metallic material such a silicon nitrate, particularly on the surface of grooves and/or depressions where it contacts the bone. Various methods to integrate such component(s) can be used, such as making a threaded sleeve of silicon nitrate material and/or surface coating some portion of the screw shaft, grooves, depressions/cups and/or thread flutes. Many methods for assembling such a design can be utilized, such as employing a horseshoe shaped sleeve which engages around a single piece central column of a pedicle screw. In various alternative embodiments, a threaded cannulated sleeve could be provided, with or without external and/or internal threaded features, and even where the base screw head and/or shaft with tip could comprise multiple components and/or multiple materials to make the assembly functional and durable. In some embodiments, a surface of the sleeve component could be configured with patterns and/or textures to further increase the surface area of bone contact within a pre-tapped hole in the bone.

Another exemplary embodiment of a surgical screw implant that incorporates silicon nitride features can include a modular sleeve and/or surface coating that enhances osseous integration and/or improves bacterial resistance. Desirably, the screw body would comprise a metallic material such as titanium, which is a commonly accepted and highly tested medical material for bone screws. However, because metal bone screws may not contribute significantly to osseous fixation, a modular sleeve can comprise a material such as silicon nitride or similar materials that desirably induce osseous integration. Such an arrangement allows silicon nitride to be integrated into the metal bone screw without sacrificing significant strength and/or durability of the screw. Alternatively, a coating of silicon nitride could be applied to one or more surfaces of the bone screw (i.e., through a laser sintering or other method), as previously described. In various alternative embodiments, Si₃N₄ powder may be laser sintered to titanium or PEEK base materials.

In various embodiments, silicon nitride can be manufactured into various shapes and/or sizes, and can be attached to a shaft or other feature of a bone screw as described herein. Because silicon nitride may not be effective on a cutting surface, the cutting tip of the bone screw may desirably comprise a metal cutting tip. Moreover, because the silicon nitride material may shrink or otherwise deform during portions of the manufacturing and/or curing process, it may be desirable that the implant design features accommodate potential changes in the design of the insert or similar components. In at least one alternative embodiment, silicon nitride material may be manufactured in a sleeve or other shape, with the corresponding metal screw shape subsequently being modified to accommodate the final cured shape and/or size of the silicon nitride sleeve insert. In various alternative embodiments, the sleeve insert could alternatively comprise a silicon nitride tip or “washer” placed around the screw head, or silicon nitride strips, inserts or “teeth” could be provided along the longitudinal length of the screw. Alternatively, one or more of the grooves and/or depressions/cups described herein could be lined and/or filled with silicon nitride, including possible designs where the silicon nitride fully filler the depression/groove and even extends outward of the grooves/cups to engage with surrounding bony structures.

Note that, in various alternative embodiments, variations in the position and/or relationships between the various figures and/or modular components are contemplated, such that different relative positions of the various modules and/or component parts, depending upon specific module design and/or interchangeability, may be possible. In other words, different relative adjustment positions of the various components may be accomplished via adjustment in separation and/or surface angulation of one of more of the components to achieve a variety of resulting implant configurations, shapes and/or sizes, thereby accommodating virtually any expected anatomical variation.

The various embodiments of a fixation device and/or implant disclosed herein can be configured to interact with one or two or more bone vertebrae of a spine or other anatomical locations. The spine may have any of several types of spinal curvature disorders which are sought to be treated. Examples of such spinal curvature disorders include, but need not be limited to, lordosis, kyphosis, scoliosis and/or low and/or high velocity fractures, among other pathologies.

In various exemplary scenarios, a variety of surgical tools can be used in conjunction with various implant devices utilized to fix and/or secure adjacent vertebrae that have had cartilaginous disc between the vertebrae replaced with fusion material that promotes the fusion of the vertebrae, such as a graft of bone tissue. Also, such can be accomplished even when dealing with a spinal curvature disorder (e.g., lordosis, kyphosis and scoliosis).

Of course, method(s) for manufacturing the surgical devices and related components and implanting an implant device into a spine are contemplated and are part of the scope of the present application.

While embodiments and applications of the present subject matter have been shown and described, it would be apparent to those skilled in the art that many more modifications are possible without departing from the inventive concepts herein. The subject matter, therefore, is not to be restricted except in the spirit of the appended claims.

All references, including publications, patent applications, and patents, cited herein are hereby incorporated by reference to the same extent as if each reference were individually and specifically indicated to be incorporated by reference and were set forth in its entirety herein.

The various headings and titles used herein are for the convenience of the reader and should not be construed to limit or constrain any of the features or disclosures thereunder to a specific embodiment or embodiments. It should be understood that various exemplary embodiments could incorporate numerous combinations of the various advantages and/or features described, all manner of combinations of which are contemplated and expressly incorporated hereunder.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., i.e., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventor for carrying out the invention. Variations of those preferred embodiments may become apparent to those of ordinary skill in the art upon reading the foregoing description. The inventor expects skilled artisans to employ such variations as appropriate, and the inventor intends for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A fixation screw for implanting into a bony tissue, comprising:
an elongated screw body having a main cylindrical portion, a forward end and a rear end,
the main cylindrical portion including a first thread located proximate to the forward end, the first thread extending radially outward from said main cylindrical portion, the first thread having a first thread angle, and
a plurality of helical groove portions extending into the main cylindrical portion, the plurality of helical groove portions having a first helix angle that is different than the first thread angle.

2. The fixation screw of claim 1, wherein the plurality of helical groove portions are located proximate to the rear end of the main cylindrical portion.

3. The fixation screw of claim 1, wherein the main cylindrical portion comprises a first diameter portion proximate to the forward end and a second diameter portion proximate to the rear end, a first diameter of the first diameter portion being less than a second diameter of the second diameter portion.

4. The fixation screw of claim 3, wherein a tapered region is positioned between the first diameter portion and the second diameter portion.

5. The fixation screw of claim 4, wherein at least a portion of the plurality of helical groove portions extend along a surface of the tapered region.

6. The fixation screw of claim 3, wherein the first thread does not extend along the second diameter portion.

7. The fixation screw of claim 4, wherein the first thread does not extend along the tapered region.

8. The fixation screw of claim 1, wherein the first helix angle is in an opposing direction to the first thread angle.

9. The fixation screw of claim 1, wherein at least a portion of the elongated screw body comprises silicon nitride.

10. The fixation screw of claim 1, wherein at least a portion of the elongated screw body comprises a silicon nitride surface coating.

11. The fixation screw of claim 1, wherein at least a portion of the plurality of helical groove portions contain silicon nitride.

12. The fixation screw of claim 10, wherein the silicon nitride surface coating is disposed on the elongated screw body using a laser deposition process.

13. The fixation screw of claim 10, wherein the silicon nitride surface coating is disposed on the elongated screw body using a powder deposition process.

14. A fixation screw for implanting into a bony tissue, comprising:
an elongated screw body having a main cylindrical portion, a forward end and a rear end,
the main cylindrical portion including a first thread located proximate to the forward end, the first thread extending radially outward from said main cylindrical portion, the first thread having a first thread angle, and
a plurality of depressions extending into the main cylindrical portion, the plurality of depressions positioned proximate to the rear end.

15. The fixation screw of claim 14, wherein the main cylindrical portion comprises a first diameter portion proximate to the forward end and a second diameter portion proximate to the rear end, a first diameter of the first diameter portion being less than a second diameter of the second diameter portion.

16. The fixation screw of claim 15, wherein a tapered region is positioned between the first diameter portion and the second diameter portion.

17. The fixation screw of claim 16, wherein the plurality of depressions extend along the second diameter portion and do not extend into the tapered region.

18. A fixation screw for implanting into a bony tissue, comprising:
an elongated screw body having a main cylindrical portion, a forward end and a rear end,
the main cylindrical portion including a first thread located proximate to the forward end, the first thread extending radially outward from said main cylindrical portion, the first thread having a first thread angle,
a plurality of helical groove portions extending into the main cylindrical portion, the plurality of helical groove portions having a first helix angle that is different than the first thread angle, and
a plurality of depressions extending into the main cylindrical portion, the plurality of depressions positioned proximate to the rear end.

19. The fixation screw of claim 18, wherein the first helix angle is in an opposing rotational direction to the first thread angle.

20. The fixation screw of claim 18, wherein the main cylindrical portion comprises:
a first diameter portion proximate to the forward end and a second diameter portion proximate to the rear end, a first diameter of the first diameter portion being less than a second diameter of the second diameter portion, and
a tapered region positioned between the first diameter portion and the second diameter portion, wherein at least a portion of the plurality of helical groove portions is formed into the tapered region, and the plurality of depressions are formed into the second diameter portion.
